(19) United States
(12) Patent Application Publication
(10) Pub. No.: US 2021/0244613 A1
(43) Pub. Date: Aug. 12, 2021

(54) STIMULATION METHOD, IN PARTICULAR LASER ACUPUNCTURE

(71) Applicant: Siegfried RITZBERGER, Wien (AT)

(72) Inventor: Siegfried RITZBERGER, Wien (AT)

(21) Appl. No.: 16/973,733

(22) PCT Filed: Jun. 7, 2019
(86) PCT No.: PCT/AT2019/060188

§ 371 Date: Dec. 9, 2020

(30) Foreign Application Priority Data
Jun. 11, 2018 (AT) A50463/2018

(51) Int. Cl. A61H 39/02

(52) U.S. Cl. CPC A61H 39/02; A61H 2039/005

(57) ABSTRACT

The invention relates to a method for the stimulation, in particular laser acupuncture, of a test person (1) in a specified body region (10), wherein—a number of reference stimulation points (RS₁, RS₂, RS₃) are specified with respect to a reference (R), in particular a three-dimensional reference, of the body region (10) to be stimulated, —an image (A), in particular a three-dimensional image, of the surface of the test person's (1) body region (10) to be stimulated is captured, points (P₁, P₂, P₃) are specified and/or ascertained in the captured image (A), the relative position of said points in the image (A) corresponding to the relative position of the reference stimulation points (RS₁, RS₂, RS₃) on the reference (R), —the stimulation points (S₁, S₂, S₃) to be stimulated corresponding to the points (P₁, P₂, P₃) in the image are ascertained on the surface of the body region (10), —at least one stimulation unit (4) is automatically aligned with at least one of the ascertained stimulation points (S₁, S₂, S₃) to be stimulated of the body region (10), and—the at least one stimulation point (S₁, S₂, S₃) to be stimulated is stimulated by means of the stimulation unit (4).

The invention refers to a procedure and a system for the stimulation, especially in laser acupuncture, of a subject in a region of the body specified in compliance with the patent claims 1 and 11.

Acupuncture is known to treat chronic back pain, chronic pain caused by osteoarthritis of the knee joint or, for example, headaches and chronic migraines, as an alternative to the treatment approaches of conventional medicine. Within this procedure, usually fine needles are placed on the acupuncture points of the subject, in order to obtain the alleviation of the conditions, where, for example, fewer undesirable side effects are to be expected than with drug therapies. Within the current level of technology, an alternative to applying acupuncture needles is using laser light. The advantage of this procedure is that there is no pain, as compared to the application of acupuncture needles, and the upper layers of the patient's epidermis are not damaged, and thus there's not risk of infection.

The disadvantage of the acupuncture procedure using acupuncture needles, or laser light, respectively, known within the current level of technology, is the fact that this procedure is adequate only to a limited extent to self-therapy, because the application of acupuncture needles, or the orientation of the laser light respectively, in areas of the body that are hard to access for the subject, for example on the back or ear, cannot be accomplished by the subject.

The object of the invention is therefore to provide a method that is easy to carry out or a stimulation method that is easy to use and which are suitable for self-acupuncture, so that a test person can treat himself.

The invention solves this task with a procedure of the kind mentioned in the preamble,
the number of reference stimulation points, in relation to a reference body region, in particular three-dimensional, to be stimulated, is provided,
an image, usually tridimensional, is captured on the surface of the body region that requires stimulation,
Points in the captured image are specified and/or determined, whose relative position on the image corresponds to the relative position of the reference stimulation points on the reference,
stimulation points to be stimulated are determined on the surface of the body region, and shall correspond to the points in the image,
at least one stimulation unit is automatically aligned on at least one of the determined stimulation points of the body region to be stimulated,
at least one stimulation point to be stimulated is stimulated with the use of a stimulation unit.

For a particularly simple determination of the stimulation points on the subject's body area that react efficiently to stimulation, it can be provided that, for the determination of the points:
in the captured image of the body region to be stimulated, specified structures, especially structures of the ear, need to be detected,
a correlation needs to be found between the structures detected in the image and the reference structures provided in the reference,
a transformation transferring the structure images to one of the known positions of the reference structures corresponding to the standard reference alignment needs to be established,
by using such transformation on the reference stimulation points, the points whose relative position in the image corresponds to the relative position of the reference stimulation points in the reference can be determined in the body region image.

For the exact determination of the points to be stimulated on the surface of the subject's body, it can be provided that the stimulation points to be stimulated need to be determined by using a transformation instruction, which can be especially specified through a camera model that records the image, whose relative position on the surface of the body region corresponds to the relative position of the points in the image.

In order to ensure an efficient therapy of the subject's condition, through effective stimulation, it can be provided that the determined stimulation points to be stimulated are stimulated with the help of at least one laser and/or compressed air and/or a push pin, where the laser has a power of at least <5 mW, in particular, and/or where the laser is preferably designed as a low-level-laser.

An exact recording of the subject's body surface is to be provided when the image, three-dimensional, in particular, of the surface of the body region to be stimulated is captured with the use of a camera equipped with infrared vision.

In order to ensure the exact stimulation of the stimulation point determined, it can be provided that, during the alignment of the stimulation unit on at least one of the stimulation points determined and/or during the stimulation of at least one stimulation point determined with the use of the stimulation unit:
images of the surface of the body region are captured at specified time intervals and the stimulation point present in the body region, currently stimulated, is determined
it is verified if the stimulation point currently stimulated is deviated, compared to the stimulation point that needs to be stimulated and
if a deviation is detected, the alignment of the stimulation unit is adjusted until the stimulation point currently stimulated corresponds to the stimulation point that needs to be stimulated.

In order to ensure that a subject cannot hurt himself during stimulation, it can be provided that, during the alignment of the stimulation unit and/or the stimulation of the stimulation point with the help of the stimulation unit,
the stimulation intensity, and particularly, the incidence of light on the subject's bodily region to be stimulated, need to be recorded, and it needs to be verified if the recorded incidence of light exceeds a specified light limit value, and a value exceeding the limit value is detected, the stimulation needs to be interrupted and/or
the pressure applied on the subject's body region that needs to be stimulated is recorded, and it shall be verified if the measured applied pressure exceeds a specified pressure limit, and if a value exceeding the limit value is detected, the stimulation needs to be interrupted.

In order to automatically adapt the stimulation to the subject's therapy necessities, it can be provided that, during the stimulation of the stimulation point, at least one of the subject's physiological and/or neurological parameters, particularly blood pressure and/or the electrophysiological signals, needs to be recorded with the help of a stimulation unit and
to verify at specified times during stimulation, if the recorded biological exceeds the upper and lower limit of a specified limit value and/or a specified range of values.
If a value exceeding the upper or lower limit is detected, the stimulation of the stimulation point needs to be adapted accordingly.

In order to increase the effectiveness of therapy, and of the stimulation, respectively, it can be provided that, during the aligning of the stimulation unit and/or the stimulation of the stimulation point with the help of a stimulation unit,
noises, sounds and/or instructions are to be emitted for the subject and/or
medicine is to be released on the stimulation point to be stimulated.

In order to ensure an effective method of stimulation, in particular for the subject's ear self-acupuncture, it can be provided that
the subject's body region to be stimulated is the ear,
as a reference, in relation to which several reference stimulation points are provided, a tridimensional ear reference is particularly provided and
the structures specified and the reference structures provided in the reference relate to ear structures.

A stimulation system complying to the invention for the stimulation, using, in particular, laser acupuncture, of a subject, in a specified body region, especially the ear, provides:
a supporting device, especially a helmet for fixing
   a recording unit, in particular a folding recording unit, for capturing an image, in particular a tridimensional image, of the surface of the body region to be stimulated,
   at least one stimulation unit for the stimulation of at least one stimulation point that needs to be stimulated on the surface of the body region and
   at least one positioning unit connected to the stimulation unit, for the aligning of the stimulation unit on the stimulation point to be stimulated,
      on the subject's body area that needs to be stimulated, where the recording unit, at least one stimulation unit and a least one positioning unit are located on the supporting device and
a processing unit connected, for data transfer purposes, to the recording unit, stimulation unit and positioning unit, where the processing unit (6) is designed for this purpose,
   the recording unit for the capturing of images of the body area to be stimulated and, eventually for activating the folding and receiving and processing images captured by the recording unit,
   to activate the positioning unit in order to align the stimulation unit over a stimulation point that needs to be stimulated, where:
      the processing unit can be provided with specific points in the recorded image of the body region and/or the processing unit is designed to determine the points of the body area in the captured image, through the specification of several reference stimulation points in relation to a reference, a tridimensional reference, in particular, whose relative position in the image corresponds to the relative position of the reference stimulation points in the reference and
      the processing unit is designed to determine the stimulation points that need to be stimulated, whose relative position on the surface of the body region corresponds to the relative position of the points in the image, and
   activates the stimulation unit to stimulate at least one determined stimulation point.

In order to very accurately determine the points on the subject's body, that are known to be therapeutically efficient, it can be provided that the processing unit needs to be designed in such a manner that it detects
images with the specified structures, especially the ear structures, in order to determine the points on the captured image of the body area that needs to be stimulated,
to find matches between the structures detected in the image and the reference structures provided in the reference,
to establish a transformation that transfers the images of structures to one of the known positions of the reference structures, which corresponds with the standard alignment provided in the reference,
by using the transformation on the reference stimulation points, to determine in the image of the body region, the points whose relative position in the image corresponds to the relative position of reference stimulation points provided in the reference.

In order to determine the actual location of points to be stimulated on the surface of the subject's body, it can be provided that the processing unit needs to be designed so that it can determine, with the help of a transformation instruction, which can be specified especially with the use of a camera model that records image, to which relative position on the surface of the body region corresponds the relative position of the points in the image.

For efficient and low-irritation stimulation of the subject, it can be provided that at least one stimulation unit comprises at least one laser and/or at least one compressed air nozzle and/or at least one pressure pin, where
the laser or the compressed air nozzle are installed in a headphone shell of the supporting device
the laser has a power of <5 mW and is preferably designed as a low-level-laser.

The creation of an exact image of the patient's body surface can be ensured if the recording unit includes a camera, in particular an infrared camera, and is especially installed in a headphone shell of the supporting device.

In order to ensure the precise stimulation of the stimulation point determined on the subject's body surface, it can be provided that the processing unit needs to be designed to
activate the recording unit at predefined time intervals, for image recording,
determine, in the captured images, the stimulation point currently stimulated on the body surface with the help of a stimulation unit,
check if the stimulation point currently stimulated is deviated, compared to the stimulation point that needs to be stimulated and
if a deviation is detected, the positioning unit needs to be activated, to align the stimulation unit, until the stimulation point, currently stimulated, corresponds to the stimulation point that needs to be stimulated.

In order to provide a highly compact structured stimulation system, it can be provided that the processing unit is located on the supporting device, in particular in a headphone shell on the supporting device.

The option of remotely managing and adjusting the stimulation order can be provided, if the processing unit is connected to an external data communication device equipped with a screen and a display unit, in particular a touchscreen display for data communication, where the processing unit
can be provided with the reference stimulation points, in relation to the reference on the external data communication device and/or
can be provided with the points on the registered image of the body surface and/or
can be provided with stimulation points on the captured image, for the purpose of correcting the points previously determined by the processing unit.

In order to mitigate the risk of injury in case of self-therapy, it can be provided that in the supporting device, especially in a headphone shell on the supporting device,
a light sensor, connected for data communication purposes to the processing unit, is installed,
   with the light sensor being designed to register the incidence of light when the holding device is fixed on the subject to be stimulated and the processing unit being designed to determine whether the detected incidence of light exceeds a predetermined light limit value, and if it detects a value exceeding the limit value, to activate the stimulation unit in order to stop the stimulation and/or
a pressure sensor connected for data communication purposes to the processing unit, is installed
   with the pressure sensor being designed to measure the contact pressure when the holding device is fixed on the subject's body region to be stimulated, and the processing unit is designed to determine whether the measured contact pressure exceeds the lower limit of the predetermined pressure limit value and, if the exceeding of the limit value is detected, to activate the stimulation unit and end the stimulation.

In order to adapt the stimulation efficiency to the subject's needs, it can be provided that the stimulation system needs to include a device for the recording of at least one of the subject's physiological and/or neurological parameters, particularly blood pressure and/or electrophysiological signals, which can be located on the subject's body, being connected, for data communication purposes, to the processing unit, wherein the processing unit is designed to
verify, in specific moments during stimulation, if the recorded biological parameter exceeds the upper and lower limits of a specified limit value and/or a specified range of values and verify, at specific moments during stimulation, if the recorded biological parameter exceeds the upper and lower limits of a specified limit value and/or a specified range of values
and, if a value exceeding the upper or lower limit is detected, the stimulation needs to be adapted, while the stimulation unit needs to be adequately activated.

In order to increase the effectiveness of the therapy, and of the subject's stimulation, respectively, it can be provided that the stimulation system
needs to include a speaker, for issuing noises, sounds and/or instructions for the subject and/or
at least one application device for the application of medicine on the stimulation point that is to be stimulated on the subject's body area.

Other advantages and configurations of the invention result from the description and the attached drawings.

Particularly advantageous, but not limiting, exemplary embodiments of the invention are shown schematically below, with reference to the accompanying drawings, and are described by way of example based on the drawings.

The following are schematically presented below:

FIG. 1 installation of a stimulation system, according to the invention, on the subject's body area to be stimulated,

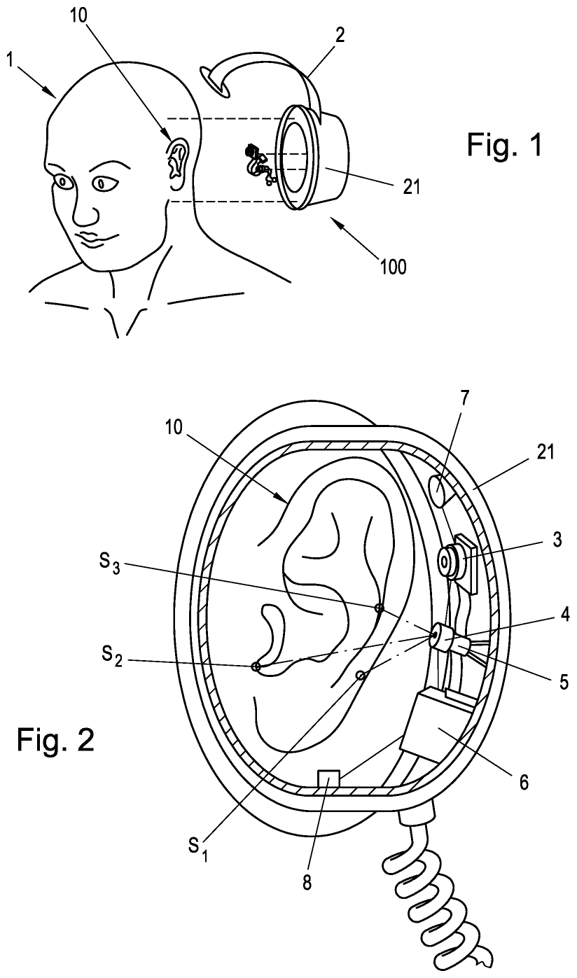

FIG. 2 an exemplary embodiment of a stimulation system, according to the invention and

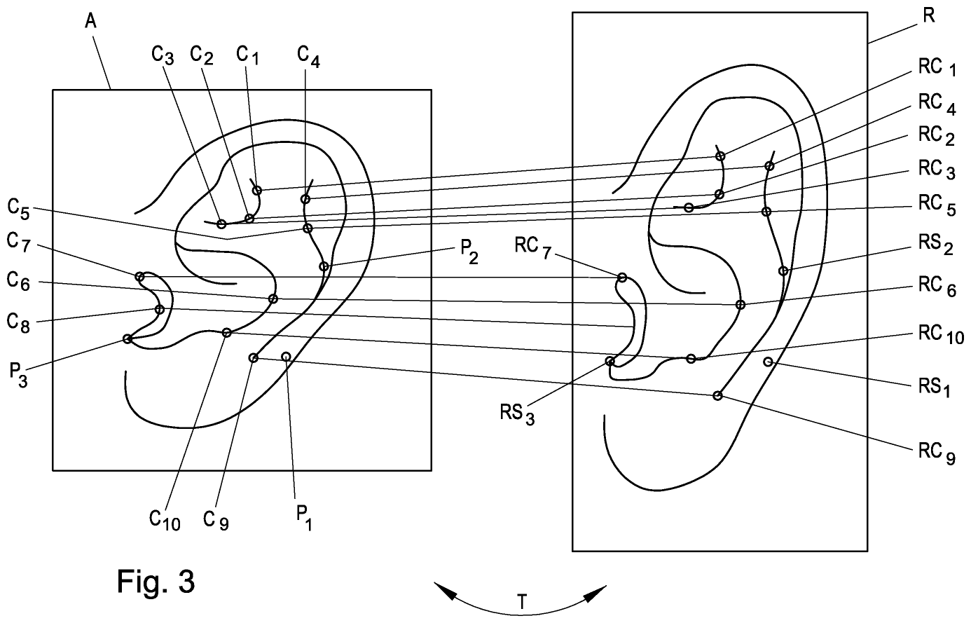

FIG. 3 transformation creation schematic.

FIG. 1 schematically presents a subject 1 that wishes to perform a self-acupuncture in a specified body area 10. In this embodiment, the specified body area 10 is the ear of subject 1. In order to be able to perform a stimulation, namely an acupuncture on the stimulation points, without external help, in the specified body area 10, subject 1 uses, in the provided embodiment, a stimulation system 100, according to the invention, which comprises a supporting device 2 and a headphone shell 21. The components necessary for the stimulation points S₁, S₂, S₃ on the ear of subject 1, which have been determined for stimulation purposes, are installed, in the provided embodiment, in the headphone shell 21 of the supporting device 2.

FIG. 2 presents a detailed view of the supporting device 2, namely of the headphone shell 21 of the supporting device 2, presented in FIG. 1. FIG. 2 presents a section of the headphone shell, which is positioned in the body area to be stimulated 10 of the subject 1.

In the provided embodiment of a stimulation system, according to 100, in the supporting device 2, in particular the headphone shell 21 of the supporting device 2, there is a recording unit 3, in particular a folding recording unit, for capturing an image A, in particular a tridimensional image, on the surface of the body area 10 to be stimulated. The recording unit 3 can be optionally designed as foldable, so that the recording unit 3 can be tilted, or folded, respectively, for space saving reasons, for example in the headphone shell 21 of the supporting device 2, when the capturing of images A is not required.

The supporting device 2 is also equipped with a stimulation unit 4, for the stimulation of at least one stimulation point S₁, S₂, S₃ on the surface of the body area 10 which needs to be stimulated. Furthermore, the supporting device 2 is equipped with at least one positioning unit 5 connected to the stimulation unit 4 for the aligning of the stimulation unit 4 to at least one stimulation point S₁, S₂, S₃ that needs to be stimulated.

As can be seen in the embodiment presented in FIG. 2, the supporting device 2 serves, in this case, for the fixing of the recording unit 3 of the stimulation unit 4, and of the positioning unit 5, on the body area 10 of subject 1, which needs to be stimulated.

The embodiment presented in FIG. 1 and FIG. 2 of a stimulation system 100, according to the invention, also comprises a processing unit 6, which is connected for data communication purposes to the recording unit 3, to the stimulation unit 4 and to the positioning unit 5. The processing unit 6 is designed to activate the recording unit to capture images of the surface of the body area that needs to be stimulated, and eventually to activate the folding and receive and process the images A captured by the recording unit 3.

The processing unit 6 also serves for the activation of the positioning unit 5 for the purpose of aligning the stimulation unit 4 to at least one stimulation point S₁, S₂, S₃ that needs to be stimulated.

For this purpose, 6 points P₁, P₂, P₃ can be previously indicated in the captured image A of the body area 10, and the processing unit 6 determines the stimulation points S₁; S₂, S₃, which need to be stimulated, if their relative position on the surface of the body area 10 of subject 1 corresponds to the relative position of points P₁; P₂, P₃ in image A.

Additionally, or alternatively, the processing unit 6 is designed to determine, by specifying a number of reference stimulation points RS₁, RS₂, RS₃ in relation to a reference, particularly a three-dimensional reference, R of the body area 10 to be stimulated, points P₁; P₂, P₃ in the captured image A, and whether their relative position in image A corresponds to the reference stimulation points RS₁; RS₂, RS₃ in reference R. In this case as well, the processing unit 6 determines the stimulation points S₁; S₂, S₃, which need to be stimulated, and whether their relative position on the surface of the body area 10 corresponds to the relative position of points P₁; P₂, P₃ in image A.

The processing unit 6 is also designed to activate the stimulation unit 4 in order to stimulate at least one of the determined stimulation points S₁; S₂, S₃.

In the example of the embodiment presented in FIG. 2, the processing unit 6 is located on the supporting device 2, namely in the headphone shell 21 of the supporting device 2. Alternatively, the processing unit 6 can be installed, for example, in an external data communication device, wherein the data communication device includes, for example, a screen and a display unit.

FIG. 2 shows that in the embodiment, the headphone shell 21 fully covers the subject's ear and sits around the ear, on the surface of the subject's body 1. Furthermore, FIG. 2 shows that the stimulation system 100 is connected through a cable to an external power supply. As an alternative, the supporting device 2, namely the headphone shell 21 may have an integrated power supply, e.g., batteries or an accumulator.

In the embodiment presented, the supporting device 2 includes only a headphone shell 21 with integrated components for the stimulation of the body region 10, namely the ear of subject 1. Alternatively, the stimulation system 100 might still include a supporting device 2 with two headphone shells 21, wherein at least in one, possibly in both headphone shells 21, there are components for the stimulation of the specified body area 10 of subject 1.

Subject 1 can, using the stimulation system 100 according to the invention, carry out, independently and without external help, a stimulation of a specified body area, particularly of his ear.

In a procedure complying with the invention for the stimulation, especially using laser-acupuncture, of a specified body area 1, it is first indicated a number of reference stimulation points RS₁; RS₂, RS₃ in relation to a three-dimensional reference R of the body area to be stimulated 10. Such a reference can be, for example, a reference image of the specified body area 10 or a three-dimensional model of the specified body area 10, wherein the reference stimulation points RS₁, RS₂, RS₃, relevant for the therapy of different disease, are specified and arranged.

In the next stage of the procedure, an image A, in particular a three-dimensional image, of the body area 10 of subject 1, which needs to be stimulated, is captured. Points P₁, P₂, P₃ are specified and/or determined successively on the captured image, their relative position on the image of the relative position of the reference stimulation points RS₁, RS₂, RS₃, corresponding to reference R.

For capturing the image, in particular the three-dimensional image, of the surface of the body area 10 to be stimulated, of subject 1, the embodiment provides a recording unit 3, in particular an infrared camera. Such a configuration of the recording unit 3 is very advantageous if the recording unit 3 is placed in a headphone shell 21. If the supporting device 2, the recording unit 3, the stimulation unit 4 and the positioning unit 5, respectively, located in the supporting device 2, are fixed on the body area 10 of subject 1, which needs to be stimulated, no outside light will enter the headphone shell 21 of the supporting device 2, therefore the use of an image recording unit for capturing images A in the visible light will not be possible.

The recording unit 3 might also alternatively include a row or surface sensor for capturing images A on the surface of the body area 10. As an alternative, the recording unit 3 can, for example, measure the distance from the specified body area 10 of subject 1, with the help of optical coherence tomography, through the light of a short length of coherence, with the help of an interferometer, and thus compose a three-dimensional image of the body area 10. As an alternative, the recording unit can also be designed to compose an image A, in particular a three-dimensional image, with the help of an electro-optical distance measurement, according to the Lidar principle, wherein, for example, pulses of laser light are emitted and their two-way transit time between the light source and the reflector, i.e., the body region 10, is used to determine the distance.

In order to determine points P₁; P₂, P₃, if their relative position in the captured image A corresponds to the relative position of the reference stimulation points RS₁; RS₂, RS₃ in reference R, in the presented embodiment of the processing unit 6, first, images of the specified structures Ci, . . . , C₁₀, which can be also found in the reference structures RC₁, . . . , RC₁₀, are determined in the captured image an A of the surface of the body area 10 to be stimulated. The specified structures Ci, . . . , C10 and the reference structures RC₁, . . . , RC₁₀, respectively, refer, for example, to characteristic structures, namely protrusions or depressions of the ear.

For this purpose, for example, a pattern recognition algorithm known from the prior art (see, for example Paul Viola and Michael Jones, Rapid Object Detection using a Boosted Cascade of Simple Features, Conference on Computer Vision and Pattern Recognition, 2001), is used to determine that the body area 10 to be stimulated is, for example, the ear of subject 1. Furthermore, by means of pattern recognition, it is determined whether it is the left or right ear of subject 1, in order to provide a subsequent comparison with the specified reference R of the body area 10.

In order to detect the characteristic structures C₁; . . . , C₁₀ in image A of the body area to be analysed 10, for example the ear of subject 1, a pattern recognition algorithm known from the prior art (see e.g., John Canny, A Computational Approach to Edge Detection, IEEE Transactions on Pattern Analysis and Machine Intelligence, Vol. PAMI-8, No. 6, November 1986) is used.

For this purpose, image areas for which in image A flat areas are differentiated along a straight or curved line, for example with a different brightness, are detected. In this case, the detection of characteristic structures C₁; . . . , C₁₀ is possible, in a very efficient manner, if the ear of subject 1 has no jewelry, piercings holes or hair.

Correlations between this type of structures C₁, . . . , C₁₀, detected in image A and the reference structures RC₁; . . . , RC₁₀ specified in reference R are successively detected, and a transformation T that transfers in standard alignment the images of structures C₁; . . . , C₁₀ to a known position of the reference structures RC₁; . . . , RC₁₀ in reference R is established. Thusly, it is possible to transfer differently shaped ears belonging to different subjects 1 to a standard alignment, in which the images of structures C₁; . . . , C₁₀ detected correspond to the known position of reference structures RC₁; . . . , RC₁₀ in reference R.

By applying the T transformation on the reference stimulation points RS₁, RS₂, RS₃, the P₁, P₂, P₃ points in image A of the body area 10, whose relative position in image A corresponds to the reference stimulation points RS1, RS₂, RS₃ in the reference R, are determined.

FIG. 3 presents an embodiment for determining points P₁; P₂, P₃ in the captured image A. In the embodiment presented, the captured image A of the body area 10 m is a two-dimensional image recording of the ear of subject 1. In the embodiment presented, the reference R of the specified body area 10 is a two-dimensional image of a reference ear. The reference R provides three reference stimulation points RS₁; RS₂, RS₃, for the treatment of certain disease symptoms.

In the embodiment presented in FIG. 3, the provided structures C₁; . . . , C₁₀ and the reference structures RC₁; . . . , RC₁₀ of reference R refer, for example, to the auditory canal, as well as to the characteristic prominences, and recesses of the ear pavilion, for example the ear helix, ear antihelix or ear cavity.

By applying the determined T transformation to the reference stimulation points RS₁, RS₂, RS₃, it is further possible, as presented in FIG. 3, to determine points P₁, P₂, P₃, which correspond to the stimulation points RS₁, RS₂, RS₃ in image A, even if the body area 10 to be stimulated, namely the ear to be stimulated of 1, is shaped differently than the reference R of the specified body area 10.

In order to determine the actual position of the stimulation points determined to be stimulated S₁, S₂, S₃ in the body area 10 of subject 1, namely his ear, a specified transformation instruction is used on points P₁, P₂, P₃ in image A. In this case, the specified transformation instruction provides the stimulation points S₁; S₂, S₃, which need to be stimulated, and whether their relative position on the surface of the body area 10 corresponds to the relative position of points P₁; P₂, P₃ in image A.

The transformation instruction specified refers, for example, to a camera model of the camera capturing image A. If the camera model, namely the camera's parameter, is known, the initial position of the point corresponding to the object in space can be determined for each image point in image A, which was captured using a recording unit 3, as an image point, for example in the two-dimensional image A.

After that, a stimulation unit 4 is automatically aligned on at least one of the stimulation points S₁, S₂, S₃, previously determined on the surface of the body area 10. In the end, at least one of the stimulations points S₁, S₂, S₃, that need to be stimulated will be stimulated with the help of the stimulation unit 4.

For the stimulation of at one of the stimulations points S₁, S₂, S₃, that need to be stimulated, at least one laser can be used as the stimulation unit 4, in compliance with the embodiment presented in FIG. 1 and FIG. 2. As an alternative, a stimulation using compressed air can be applied, case in which, on the supporting device 2 of the stimulation system 100 of the invention, at least one compressed air nozzle is installed. A stimulation system 100 that complies to the invention, can include at least one stimulation unit 4, instead of a laser and laser arrays, which can be advantageously aligned on the selected stimulation points S₁; S₂, S₃.

As an alternative, a stimulation system 100 complying to the invention for the stimulation of at least one stimulation point S₁; S₂, S₃ that needs to be stimulated, may also include, for example, water nozzles, from which a water jet is automatically oriented on at least one of the stimulation points S₁; S₂, S₃. Alternatively, a stimulation system 100 according to the invention might also include acupuncture needles or pressure pins, which are automatically applied to at least one of the stimulation points S₁; S₂, S₃, or are oriented on at least one of the stimulation points S₁; S₂, S₃.

For the stimulation of the stimulation points S₁; S₂, S₃ with the help of at least one laser, a low intensity laser suffices, therefore a laser with a power lower than 5 mW or a low-level-laser can be used. This increases the safety of use.

In this context, automatically aligned means that, during a procedure carried out with the stimulation system 100 presented in FIG. 2, the processing unit 6 activates the positioning unit 5, directs the stimulation unit 4 on at least one of the stimulation points S₁; S₂, S₃, so that the subject 1 does not have to perform or to control this operation by himself. Thus, the automated selection of the stimulation points S₁; S₂, S₃ that need to be stimulated and the automated alignment of the stimulation unit 4 or of the laser on the stimulation points are ensured, so the subject can apply the therapy himself, without effort.

In an advantageous embodiment of the invention, the processing unit 6 is connected for data communication purposes to an external data communication device equipped with a screen and a display unit, e.g., a touch display, for example a smartphone or a tablet. The data communication connection can be achieved with the use of a network or fibre optic cable, but also wireless, e.g., through a radio connection. With the help of an external data communication device, it is possible to specify the reference stimulation points RS₁; RS₂, RS₃ in relation with the reference R and/or points P₁; P₂, P₃ in the captured image A of the body area 10 and/or to specify details for the correction of points P₁; P₂, P₃ to be stimulated, which have previously been determined by the processing unit 6 in the registered image.

If, for example, the processing unit 6 cannot determine in image A any such points P₁; P₂, P₃ that correspond to the reference stimulation RS₁; RS₂, RS₃ in reference R, the processing unit 6 is designed to send an error message to subject 1, information which will be displayed on the display unit of the external data communication device.

This configuration of a stimulation system 100 according to the invention which comprises an external data communication device, provides subject 1 with the possibility of independently selecting points P₁; P₂, P₃ in image A, for example with the use of the touchscreen, if the processing unit 6 is unable to determine the points P₁, P₂, P₃ whose relative position in image A corresponds to the relative position of the reference stimulation points RS1, RS₂, RS₃ in reference R.

For this purpose, the reference R and image A can be displayed on the external data communication device's screen. This configuration of the stimulation system 100 according to the invention, gives subject 1 the possibility of controlling and correcting, since he can, for example, display and verify points P₁; P₂, P₃ in image A, which have been determined by the processing unit 6, and, if necessary, adapt the position of points P₁; P₂, P₃ in image A.

This configuration also offers to possibility of remotely managing and adjusting the stimulation with the help of a stimulation system 100, in which case, a physician monitoring the self-therapy of subject 1 can specify reference stimulation points RS1, RS₂, RS₃ in relation to reference R and/or, for example, the physician can specify points P₁, P₂, P₃ in the captured image A of the body area 10 and/or correct points P₁, P₂, P₃ determined by the processing unit in image A.

This configuration of a stimulation system 100 according to the invention also offers the possibility to, for example, sent the image A of the ear of subject 1 to a data base, to be provided as reference R with the reference stimulation points RS₁; RS₂, RS₃, and be subsequently accessed as reference R for subject 1 or for other subjects.

In the described embodiment of a stimulation system 100 according to the invention, the processing unit 6 is designed to activate the recording unit 3 at specified time intervals, for capturing images A of the body area 10 and determine, in the captured images A, the stimulation point that needs to be stimulated on the surface of the body area 10, with the help of the stimulation unit 4.

After that, the processing unit 6 will verify if the stimulation point currently stimulated is deviated in relation to the stimulation point S₁, S₂, S₃ that needs to be stimulated. In case of a deviation, the processing unit 6 will control the positioning unit 5 to align the stimulation unit 4, until the alignment of the stimulation unit 4 is duly adapted, so that the currently stimulated stimulation points corresponds to the stimulation point S₁, S₂, S₃ that needs to be stimulated.

This procedure is very advantageous during the alignment of the stimulation unit 4 to at least one of the stimulation points S₁, S₂, S₃ determined and/or or during stimulation, to check on at least one stimulation point S₁, S₂, S₃ determined with the help of the stimulation unit 4, whether the chosen stimulation point S₁, S₂, S₃ is, for example, irradiated with laser. If this is not the case, that the alignment of the stimulation unit 4 is corrected until it finds the stimulation point S₁, S₂, S₃ that needs to be stimulated, thus increasing the therapy efficiency.

The positioning unit 5 for the aligning of a stimulation unit 4, can mean an optomechatronic system, in which the stimulation unit 4, namely the laser, is positioned on a rotating disk, so that the laser is rotating, and folding, respectively. The stimulation unit 4 is additionally moved, for example, through two linear servo motors, moved in the supporting device 2, namely in the headphone shells 21 in the supporting device 2, so that they can be aligned in an optimal position for irradiation, namely for the stimulation of a stimulation point S₁, S₂, S₃. As an alternative, the positioning unit 5 for the aligning of the stimulation unit 4 can be designed as an azimuth control system. Alternatively, the stimulation unit, at least one, can be electronically aligned.

In order to increase the safety of service, and of use, respectively, the stimulation system 100 can also be equipped with an optical sensor and/or a pressure sensor 8, if necessary for the embodiment presented in FIG. 2

In the presented embodiment, an optical sensor 7 is placed in the headphone shells 21 of the supporting device. The optical sensor is connected for data communication purposes with the communication unit 6 and is designed to register the light incidents upon the fixing of the supporting device 2 on the body region to be stimulated 10 of the subject. The processing unit 6 verifies if the light incidence exceeds the specified limit value and, if it detects the exceeding of a limit value, it controls the processing unit 6, the stimulation unit 4, to end the stimulation.

This embodiment of a stimulation system 100 according to the invention is very advantageous for preventing a subject 1 from damaging his eye, e.g., with the laser light or with compressed air, when pulling off the supporting device 2 to which the stimulation components are attached. If the supporting device 2 is fixed on the body region to be stimulated 10, no external light comes into the headphone shell 21, so that a light incidence determined by the optical sensor 7 indicates the fact that the supporting device 2 and the stimulation need to be interrupted with the help of the stimulation unit 4.

In addition, or as an alternative to the optic sensor 7, a pressure sensor 8 can be placed on the supporting device, especially in the headphone shell 21, which is connected for data communication purposes with the processing unit 6. In the embodiment of FIG. 2 the pressure sensor 8 is placed advantageously in the lining of the headphone shell 21, which is attached on the supporting device 2, on the body region to be stimulated 10 on the body surface of subject 1. The pressure sensor 8 is designed to measure the pressure upon the fixing of the supporting device 2 on the body region 10 that needs to be stimulated and the processing unit 6 verifies if the pressure exceeds a specified limit.

If the pressure exceeds the lower value of a pressure limit, this indicates that the supporting device 2 was removed from the body area 10 that needs to be stimulated, and the stimulation needs to be ended in order to exclude a risk of injury. Upon the detection of a value exceeding the lower value of the limit, the processing unit 6 controls the stimulation unit 4 to end the stimulation.

Optionally, the stimulation system 100 according to the invention might include at least one device, which can be especially applied on the body of subject 1, for registering at least one biological parameter of subject 1, which is connected for data exchange purposes with the processing unit 6. The biological parameter might refer to the blood pressure, pulse, EEG or EKG values of subject 1, but also to the skin resistance, skin conductivity, temperature and respiration rate of subject 1.

The processing unit 6 is designed to verify, at specified times during stimulation, if the registered biological parameter exceeds the upper and lower limit of a specified value and/or range of values and verify, at pre-established moments during stimulation, whether the registered biological parameter exceeds the inferior or superior limit of a specified value and/or range of values. If this occurs, then it indicates that the stimulation of at least one stimulation point S₁; S₂, S₃ on the body surface 10 of subject 1 is, for example, to intense, and the subject perceives the stimulation as uncomfortable, or that the stimulation is to weak, thus generating a therapy with reduced effect on subject 1.

If a value exceeding the lower or upper value of a limit value or of a specified range of values, the processing unit 6 commands the stimulation unit 4 adequately, and adapts the stimulation, namely the laser acupuncture, of the stimulation point S₁, S₂, S₃ that needs to be stimulated in order to obtain an optimal therapeutic effect for subject 1. In this case, the connection duration and intensity of the laser light are adapted to the therapeutic requirements of subject 1.

In order to amplify the therapeutic effect of the stimulation, namely of the laser acupuncture, on a subject 1, a stimulation system 100, according to the invention, can optionally comprise a speaker for issuing noises, sounds and/or indications for the subject 1, which is preferably located in a headphone shell 21 of the supporting device 2.

A stimulation system 100 complying to the invention may comprise at least one application device for the application of medicine on the stimulation point S₁; S₂, S₃ that needs to be stimulated on the surface of the body region 10 of subject 1. The application device serves, for example, to spray or apply with a brush medicine that acts in a specific manner when the laser light is applied, thus enhancing the therapeutic effect on subject 1 on the body areas, namely the ear.

A stimulation system 100, according to the invention, may also include goggles for issuing light impulses or images for subject 1, due to which the brain waves of subject 1 are adjusted to a certain frequency and the subject enters a relaxation state, so that the stimulation, namely the laser acupuncture of the ear of subject 1 is very efficient.

1. Procedure for the stimulation, especially with laser-acupuncture, of the subject (1) in a specified body area (10)
Where the number of reference stimulation points (RS₁, RS₂, RS₃), in relation to a reference (R), especially a three-dimensional one, of the body region (10) to be stimulated, is specified,
An image (A), especially a three-dimensional one, of the body region (10) of the subject (1) that needs to be stimulated, is registered,
Points (P₁; P₂, P₃) are specified/determined in the registered image (A), their relative position in the image (A), in relation to the relative position of the reference stimulation points (RS₁, RS₂, RS₃), corresponding to the reference (R),
The stimulation points (S₁; S₂, S₃) that need to be stimulated are determined on the surface of the body region (10), corresponding to the points (P₁; P₂, P₃) in the image,
At least one stimulation unit (4) is automatically centred on at least one of the determined stimulation points (S₁, S₂, S₃) of the body region (10) that needs to be stimulated,
In the end, at least one stimulation point (S₁, S₂, S₃) that needs to be stimulated, will be stimulated with the help of a stimulation unit (4).

2. Procedure complying to claim 1, characterized in particular by the fact that, in order to determine the points (P₁, P₂, P₃)
Specified structures (C₁, . . . , C₁₀), especially ear structures, need to be detected in the captured image (10) of the surface of the body region (10) which needs to be stimulated,
The correspondences between the structures (C₁; C₁₀) detected in the image (A) and the specified reference structures (RC₁; . . . , RC₁₀) of the reference (R) need to be found,
A transformation (T) transferring the images of structures (C₁; C₁₀) to one of the known positions of the reference structures (RC₁; RC₁₀) which correspond to the standard alignment in the reference (R) needs to be established,
The application of transformation (T) on the reference stimulation points (RS₁; RS₂, RS₃) determines the points (P₁; P₂, P₃) in image (A) of the body area (10), whose relative position in the image (A) corresponds to the reference stimulation points (RS₁, RS₂, RS₃) in the reference (R).

3. The procedure according to claim 1 is characterized by the fact that the stimulation points that need to be stimulated (S₁, S₂, S₃), whose relative position on the surface of the body area (10) corresponds to the relative position of the points (P₁, P₂, P₃) in image (A), are determined through the use of a specified transformation, especially though a camera model, which can be specified in relation to the camera recording the image (A).

4. Procedure according to claim 1, characterized in that the determined stimulation points (S₁, S₂, S₃) are stimulated with the help of at least one laser or/compressed air and/or at least one pressure pin, where the laser has a power of at least <5 mW, especially when the laser is preferably designed as a low-level-laser.

5. Procedure according to claim 1, characterized in that the image (A), especially a three-dimensional one, of the body area to be stimulated (10) is captured with the help of a camera, especially an infrared camera.

6. Procedure according to claim 1, characterized in that the during the alignment of the stimulation unit (4) in at least one of the determined stimulation points (S₁, S₂, S₃) and/or during the stimulation of at least one stimulation point (S₁, S₂, S₃) determined, with the help of a stimulation unit (4)
Captures images (A) of the body region surface (10) at specified time intervals and determines the stimulation point of the body region (10), which is currently stimulated
It is verified whether the stimulation point (S₁, S₂, S₃) that is currently stimulated deviates from the stimulation point that needs to be stimulated and
If a deviation is detected, the alignment of the stimulation unit (4) is adjusted until the stimulation point currently stimulated corresponds to the stimulation point (S₁, S₂, S₃) that needs to be stimulated.

7. Procedure according to claim 1, characterized in that the during the alignment of the stimulation unit (4) and/or the stimulation of the point to be stimulated (S₁, S₁, S₂) with the help of a stimulation unit (4)
The intensity of the stimulation is recorded, in particular the light incidence on the body region (10) of the subject (1), which needs to be stimulated; it is verified if the light incidence exceeds a specified light limit value, and if a value exceeding the limit value is detected, the stimulation needs to be stopped and/or
the pressure applied to the body area to be stimulated (10) of the subject (1) is registered, verified, if the measured applied pressure exceeds the lower limit of a specified pressure limit value, and if a value exceeding such limit value is detected, the stimulation is stopped.

8. Procedure according to claim 1, characterized by the fact that during the stimulation of the stimulation point (S₁, S₂, S₃) determined with the help of the stimulation unit (4), at least one physiological and/or neurological parameter of the subject (1) is recorded, especially blood pressure and/or electrophysiological signals and
It shall be verified at provided moments during stimulation whether the registered biological parameter exceeds or not the upper and lower limit of a specified limit value and/or of a specified value range.
In case of detecting a value exceeding the upper or lower limit, the stimulation of the stimulation points (S₁, S₂, S₃) will be adapted adequately.

9. Procedure according to claim 1, characterized in that the during the alignment of the stimulation unit (4) and/or the stimulation of the stimulation point (S₁, S₂, S₃) with the help of a stimulation unit (4)
Noises, sounds and/or instructions are emitted for the subject (1) and/or
Medication is released on the stimulation point (S₁, S₂, S₃) that needs to be stimulated.

10. Procedure according to claim 1 for the stimulation, particularly the acupuncture, of a subject (1) at the ear, characterized in that
The subject's body region (10) that needs to be stimulated is the ear,
As a reference (R), in relation to which a number of reference stimulation points (RS₁; RS₂, RS₃) are specified, a three-dimensional reference (R) of the ear is specified and
of the specified structures (C₁; . . . , C₁₀) and the reference structures (RC₁; . . . , RC₁₀) specified in the reference (R) refer to ear structures.

11. Stimulation system for the stimulation, in particular using laser-acupuncture, of the subject (1), in a specified body area (10)
A supporting device (2), especially a helmet
of a recording unit (3), in particular a folding one, for the recording of an image (A), especially a three-dimensional one, of the surface of the body region (10) to be stimulated,
At least one stimulation unit (4) for the stimulation of at least one stimulation point (S₁, S₂, S₃) which needs to be stimulated on the surface of the body region (10) and
At least a positioning unit connected to the stimulation unit (4) for the alignment of the stimulation (4) on the stimulation point which needs to be stimulated (S₁, S₂, S₃),
on the body area (10) which needs to be stimulated on subject (1), where the recording unit (3), wherein at least one stimulation unit (4) and one positioning unit (5) are placed on the supporting device (2) and
And a processing unit (6) that is connected for data communication purposes to the registration unit (3), stimulation unit (4) and with the positioning unit (5) where the processing unit (6) is designed to command
The recording unit (3) for capturing images (A) on the surface of the body area (10) that needs to be stimulated and, eventually, for activating the folding and receiving and processing the images captured (A) by the recording unit (3),
To activate the positioning unit (5) for the alignment of the stimulation unit (6) on a stimulation point (S₁, S₂, S₃) that needs to be stimulated, where the following can be specified
The processing unit (6) points (P₁, P₂, P₃) in image (A) of the body area and/or the processing unit (6) is designed to determine by specifying a number of reference stimulation points (RS₁; RS₂, RS₃) in relation to a reference, in particular a three-dimensional reference (R) of the body area to be stimulated (10), the points (P₁; P₂, P₃) in the captured image (A), and whether their relative position in image (A) corresponds to the reference stimulation points (RS₁; RS₂, RS₃) in reference (R), and
The processing unit (6) is designed to determine the stimulation points (S₁; S₂, S₃) that need to be stimulated, and whether their relative position on the surface of the body area (10) corresponds to the relative position of points (P₁; P₂, P₃) in image (A) and
Activates the stimulation unit (4) to stimulate at least one determined stimulation point (S₁; S₂, S₃).

12. Stimulation system according to claim 11 characterized in that the processing unit (6) is designed to determine points (P₁, P₂, P₃)
To detect specified structures (C₁, . . . , C₁₀), especially ear structures, in the captured image (A) of the surface of the body region (10) that needs to be stimulated,
To find correlations between the structures detected (C₁, . . . , C₁₀) in the image (A) and the reference structures (RS₁; RS₂, RS₃) specified in the reference, (R)
To establish a transformation (T) transferring the images of structures (C₁; . . . , C₁₀) to one of the known positions of the reference structures (RC₁; . . . , RC₁₀), which corresponds to the standard alignment in reference (R),
By applying the transformation (T) on the reference stimulation points (RS₁; RS₂, RS₃), the points (P₁; P₂, P₃) in image (A) of the body area (10), whose relative position in image (A) corresponds to that of the reference stimulation points (RSi, RS₂, RS₃) in the reference (R) are determined.

13. A stimulation system according to claim 11, characterized in that the processing unit (6) is designed to determine, with the help of a transformation order, which can be, in particular, specified through a camera model for the camera recording the image (A), the stimulation points (S₁; S₂, S₃) that need to be stimulated, whose position on the surface of the body area (10) corresponds to the relative position of points (P₁, P₂, P₃) in image (A).

14. Stimulation system according to claim 11, characterized in that at least one stimulation system (4) comprises at least one laser and/or at least one compressed air nozzle and/or at least one pressure pin, wherein
The laser or the compressed air nozzle are installed in a headphone shell (21) of the supporting device (2)
The laser has a power of <5 mW and is preferably designed as a low-level-laser.

15. Stimulation system according to claim 11 Characterized in that the recording unit (3) comprises at least an infrared camera, in particular, and is installed, in particular, in a headphone shell (21) of the supporting device (2).

16. Stimulation system according to claim 11 Characterized in that the processing unit (6) is designed
To activate the recording unit (3) at previously established time intervals, for the capturing of images,
To determine, in the captured images (A), the stimulation point currently stimulated on the surface of the body area (10), with the help of the stimulation unit (4),
The check whether the stimulation point (S₁, S₂, S₃) that is currently stimulated is deviated from the stimulation point that needs to be stimulated and
If a deviation is detected, the positioning unit (5) needs to be activated in order to align the stimulation unit (4) until the stimulation point currently stimulated corresponds to the stimulated point (S₁, S₂, S₃) that needs to be stimulated.

17. Stimulation system according to claim 11, characterized in that the processing unit (6) is positioned in the supporting device (2), especially in a headphone shell (21) of the supporting device (2).

18. Stimulation system according to claim 11 characterized in that the processing unit (6) is connected to an external data communication device equipped with a screen and a display unit, in particular a touch-display for data communication, where the processing unit (6) can be provided with specified reference stimulation points (RS₁; RS₂, RS₃) in relation to reference (R) through the external data communication device and/or
The points (P₁, P₂, P₃) in the captured image (A) of the body region (10) can be specified and/or
The points to be stimulated (P₁, P₂, P₃), in the captured image (A), for the correction of points (P₁; P₂, P₃) previously determined by the processing unit (6), can be specified.

19. Stimulation system according to claim 11 Characterized in that the supporting device (2), is placed, in particular,
in a headphone shell (21) of the supporting device (2),
At least one optical sensor (7) that is connected for data communication purposes at least with the processing unit (6),
Where the optical sensor (7) is designed to register the incidence of light upon the fixing of the supporting device on the body area (10) which needs to be stimulated, of the subject (1)
Where the processing unit (6) is designed to check whether the measured incidence of light exceeds the lower limit of a specified light limit value and, upon detecting a value exceeding the lower limit, to activate the stimulation unit (4) and stop the stimulation and/or
At least one pressure sensor (8) that is connected for data communication purposes at least with the processing unit (6),
Where the optical sensor (8) is designed to register the incidence of light upon the fixing of the supporting device on the body area (10) to be stimulated of the subject (1)
Where the pressure unit (6) is designed to check whether the measured pressure exceeds the lower limit of a specified pressure limit and upon the detection of a value exceeding the lower limit of the limit value, to activate the stimulation unit (4) and stop the stimulation unit.

20. Stimulation system according to claim 11 characterized in that the stimulation system comprises at least one device for the recording of at least one physiological and/or neurological parameter of the subject (1), in particular blood pressure and/or electrophysiological signals, which can be placed on the subject's body (1), which is connected for data communication purposes to the processing unit (6), where the processing unit (6) is designed to
Check, at specified times during the stimulation, whether the recorded biological parameter exceeds the upper and lower limit of a specified limit value and/or of a specified range of values, and check at previously established moments during stimulation, whether the recorded biological parameter exceeds the upper and lower limit of a specified limit value and/or of a specified range of values
And in case of detecting a value exceeding the upper or lower limit, to adapt the stimulation, adequately activating the stimulation unit (4).

21. Stimulation system according to claim 11 characterized in that the stimulation system
Comprises a speaker for issuing noises, sounds and/or instructions for the subject (1) and/or
At least one device for applying medicine on the stimulation point (S₁; S₂, S₃) which needs to be stimulated on the surface of body region (10) of the subject (1).
